(19) United States
(12) Patent Application Publication
(10) Pub. No.: US 2010/0137890 A1
(43) Pub. Date: Jun. 3, 2010

(54) Fibrous Surgically Implantable Mesh

(75) Inventors: Jean-Pierre Elisha Martinez, Lod (IL); Alexander Dobson, Petach-Tikva (IL); Alon Shalev, Raanana (IL)

Correspondence Address: The Law Office of Michael E. Kondoudis, 888 16th Street, N.W., Suite 800, Washington, DC, 20006, US

(73) Assignee: NICAST LTD., Lod (IL)

(21) Appl. No.: 12/699,241

(22) Filed: Feb. 3, 2010

Related U.S. Application Data
(63) Continuation-in-part of application No. PCT/IL2008/001061, filed on Aug. 3, 2008.
(60) Provisional application No. 60/935,283, filed on Aug. 3, 2007.

(51) Int. Cl. A61B 17/03

(52) U.S. Cl. 606/151

(57) ABSTRACT

A fibrous mesh surgically implantable into a mammal internal cavity is disclosed. The aforesaid mesh has a laminar extra-cellular-like matrix structure. The mesh comprises a first layer characterized by porosity effective for mammal tissue infiltration into the first layer and a substantially non-porous second layer. The first layer is adapted to surgically adhere to a cavity wall in need of repair such that wall tissues infiltrate thereinto while the second layer is characterized by non-adhesion and adapted for non-traumatic contact to mammal viscera and omentum. The first layer is biodegradable and the second layer is tissue-integrated with the cavity wall.

### CROSS-REFERENCE TO RELATED APPLICATION

This application is a continuation-in-part of International Patent Application No. PCT/IL2008/001061, which was filed on Aug. 3, 2008, and which claims the benefit of priority from U.S. Provisional Patent Application No. 60/935,283, filed Aug. 3, 2007.

### FIELD OF THE INVENTION

The present invention relates to a surgically implantable mesh for reconstruction of hernias and soft tissue deficiencies and temporary bridging of facial defects and, more specifically, to a two-layers mesh made of polymer fibres by electrospinning.

### BACKGROUND OF THE INVENTION

A hernia is a protrusion of a tissue, structure, or part of an organ through the muscular tissue or the membrane by which it is normally contained. The hernia has three parts: the orifice through which the aforesaid hernia herniates, the hernial sac, and contents of the aforesaid sac. An untreated hernia may complicate by: (a) Inflammation; (b) Irreducibility; (c) Obstruction; (d) Strangulation; and (e) Hydrocele of the hernial sac.

Inguinal Hernia

By far the most common hernias (up to 75% of all abdominal hernias) are the so-called inguinal hernias. For a thorough understanding of inguinal hernias, much insight is needed in the anatomy of the inguinal canal. Inguinal hernias are further divided into the more common indirect inguinal hernia (⅔, depicted here), in which the inguinal canal is entered via a congenital weakness at its entrance (the internal inguinal ring), and the direct inguinal hernia type (⅓), where the hernia contents push through a weak spot in the back wall of the inguinal canal. Inguinal hernias are more common in men than women while femoral hernias are more common in women.

Femoral Hernia

Femoral hernias occur just below the inguinal ligament, when abdominal contents pass into the weak area at the posterior wall of the femoral canal. They can be hard to distinguish from the inguinal type (especially when ascending cephalad): however, they generally appear more rounded, and, in contrast to inguinal hernias, there is a strong female preponderance in femoral hernias. The incidence of strangulation in femoral hernias is high. Repair techniques are similar for femoral and inguinal hernia.

Umbilical Hernia

Umbilical hernias are especially common in infants of African descent, and occur more in boys. They involve protrusion of intraabdominal contents through a weakness at the site of passage of the umbilical cord through the abdominal wall. These hernias often resolve spontaneously. Umbilical hernias in adults are largely acquired, and are more frequent in obese or pregnant women. Abnormal decussation of fibers at the linea alba may contribute.

Diaphragmatic Hernia

Higher in the abdomen, an (internal) “diaphragmatic hernia” results when part of the stomach or intestine protrudes into the chest cavity through a defect in the diaphragm.

A hiatus hernia is a particular variant of this type, in which the normal passageway through which the esophagus meets the stomach (esophageal hiatus) serves as a functional “defect”, allowing part of the stomach to (periodically) “herniate” into the chest. Hiatus hernias may be either “sliding,” in which the gastroesophageal junction itself slides through the defect into the chest, or non-sliding (also known as para-esophageal), in which case the junction remains fixed while another portion of the stomach moves up through the defect. Non-sliding or para-esophageal hernias can be dangerous as they may allow the stomach to rotate and obstruct.

A congenital diaphragmatic hernia is a distinct problem, occurring in up to 1 in 2000 births, and requiring pediatric surgery. Intestinal organs may herniate through several parts of the diaphragm, posterolateral (in Bochdalek's triangle, resulting in Bochdalek's hernia), or anteromedial-retrosternal (in the cleft of Larrey/Morgagni's foramen, resulting in Morgagni-Larrey hernia, or Morgagni's hernia).

Ventral Hernia

Ventral hernias which are also referred as Post Operative Ventral Hernias (POVH) may occur following surgery in the abdomen, whether the surgery is an open surgery or a laparoscopy: as a result of the intervention the abdominal wall may weaken until it is not able to sustain the abdominal pressure exercised by the viscera and creates a so-called incisional hernia.

Current medical practice in hernia repair (herniorrhaphy) often involves the use of a prosthetic (surgical) mesh, to reduce tension of the healing region (“tension free technique”) and to secure the weak area under the peritoneum.

Abdominal wall hernias occur in 15-30% of patients following previous laparotomy. Laparoscopic hernia repair appears to be superior over traditional open repair in the following aspects: (1) It reduces pain and shortens hospitalization and recovery time and thus reduce lost workdays. (2) It facilitates repair of recurrent and bilateral hernia. (3) Scars are small and hardly noticeable. However, laparoscopic hernia intraperitoneal onlay mesh (IPOM) repair is dependent on the use of mesh material that can be safely placed in contact with the abdominal mesothelium and viscera without creating adhesions which in turn may lead to intestinal obstruction or even erosion of the viscera and fistula formation.

It is generally advisable to repair hernias in a timely fashion, in order to prevent complications such as organ dysfunction, gangrene, and multiple organ dysfunction syndrome. Most abdominal hernias can be surgically repaired, and recovery rarely requires long-term changes in lifestyle. Uncomplicated hernias are principally repaired by pushing back, or “reducing”, the herniated tissue, and then mending the weakness in muscle tissue (an operation called herniorrhaphy). If complications have occurred, the surgeon will check the viability of the herniated organ, and resect it if necessary. Modern muscle reinforcement techniques involve synthetic materials (a mesh prosthesis) that avoid over-stretching of already weakened tissue (as in older, but still useful methods). The mesh is placed over the defect, and sometimes staples are used to keep the mesh in place. Evidence suggests that this method has the lowest percentage of recurrences and the fastest recovery period. Increasingly, some repairs are performed through laparoscopes.

Many patients are managed through day surgery centers, and are able to return to work within a week or two, while heavy activities are prohibited for a longer period. Patients who have their hernias repaired with mesh often recover in a number of days. Surgical complications have been estimated to be up to 10%, but most of them can be easily addressed. They include surgical site infections, nerve and blood vessel injuries, injury to nearby organs, and hernia recurrence.

The new trends for hernia repair include minimal-invasive techniques, in which the hernia defect is closed by a piece of non-absorbable mesh with minimal tension—so called “tension-free” hernia repair. The follow-up times thus far are short for such procedures, but it seems that recurrence rates of 1% or below could be expected. Also, the general recovery time has become shorter, and the patients are usually encouraged to begin their normal activities with no restrictions within a week after the operation.

To function properly, the ideal prosthetic device must allow or even induce strong adhesion to the tissues of the abdominal wall. However it must be as frictionless as possible toward the visceral side, to avoid intestinal obstruction or enterocutaneous fistulae. Existing prosthetic meshes often do not meet this primary request at the satisfaction of the medical community or are difficult to handle and fix to the abdominal wall.

U.S. Pat. No. 6,319,264 ('264) discloses a flexible, fibrous hernia mesh, which is intended to be implanted to close hernia defects. The mesh has at least two functional components or layers: (1) a rapidly degradable first layer and (2) a more slowly degradable (with respect to the first layer) second layer. Using the fibrous mesh of this invention, the hernia defect can be closed so that a) the second layer supports the area until the scar tissue is strong enough (around 6 months), to prevent recurrent hernia formation, b) while the more rapid degradation of the first layer induces scar tissue formation due to inflammatory reaction, and c) the second layer isolates the first layer from the abdominal cavity, preventing tissue to tissue adhesion onto the intestines. The mesh is placed on the uncovered fascia area with its more rapidly absorbable side (the first layer) towards the fascia.

However, in accordance with '264, the implanted mesh is in traumatic contact to viscera. Thus, an unmet long-felt need is to provide a bi-functional prosthetic device that is able: (a) to be strongly adhered to the tissues of the abdominal wall and (b) to permanently non-traumatically contact to the visceral side to avoid intestinal obstruction or enterocutaneous fistulae. It should be emphasized that known in the prior art technical solutions provide only temporary solutions of abovementioned problem. There are materials (for example, Parietex Composite, see Schreinemacher M H, Emans P J, Gijbels M J, Greve J W, Beets G L, Bouvy N D. Degradation of mesh coatings and intraperitoneal adhesion formation in an experimental model. Br J Surg 2009; 96(3):305-313) which are characterized by growing adhesion to the omentum.in the post implantation period. In contrast to the prior art, the needed technical solution should comprises an ingrowth assisting the biodegradable portion of the prosthetic device attached to the abdominal wall, while a universal anti-adhesion portion should be bio-stable and adapted for tissue-support with the cavity wall

### SUMMARY OF THE INVENTION

It is hence one object of the invention to disclose a fibrous mesh surgically implantable into a mammal internal cavity. The aforesaid mesh has a laminar extra-cellular-like matrix structure. The mesh comprises a first layer characterized by a porosity effective for mammal tissue infiltration into the first layer and a substantially non-porous second layer which prevents abdominal viscera and omentum adhesions. The first layer is adapted to surgically adhere to a cavity wall in need of repair such that wall tissues infiltrate thereinto while the second layer is characterized by non-adhesion and adapted for non-traumatic contact to mammal viscera.

It is a core purpose of the invention to provide the first layer is biodegradable and the second layer is tissue-integrated with the cavity wall.

Another object of the invention is to disclose the mesh effectively elastic for non-interfering with a repaired mammal cavity wall.

A further object of the invention is to disclose the mammal which is a human.

A further object of the invention is to disclose the mesh comprising electrospun fibres.

A further object of the invention is to disclose the electrospun fibers which are of nanometric size.

A further object of the invention is to disclose the first layer made of a material selected from the group consisting of polyurethane, collagen, fibrin, fibronectin, vitronectin, laminin, protein further comprising cellular adhesion peptides, protein comprising CDPGYIGSR (Cys-Asp-Pro-Gly-Tyr-Ile-Gly-Ser-Arg) peptide linked polymer, arginine-glycine-aspartic acid peptide linked polymer, RGDS (arf-gly-asp-ser) peptide linked polymer, YIGSR (Tyr-Ile-Gly-Ser-Arg) peptide linked polymer, and any combination thereof.

A further object of the invention is to disclose the protein comprising at least one of component selected from the group consisting of arginine-glycine-aspartic acid-rich sequences, RGDS (arf-gly-asp-ser)-rich sequences, YIGSR (Tyr-Ile-Gly-Ser-Arg)-rich sequences, CDPGYIGSR (Cys-Asp-Pro-Gly-Tyr-Ile-Gly-Ser-Arg)-rich sequences and any combination thereof.

A further object of the invention is to disclose the second layer made of a material selected from the group consisting of polytetrafluorethylene, fluor based polymer, polyvinylidene fluoride, a hydrophobic material, polyester, polypropylene, polyformaldehyde, silicone rubber, poly(ethylene glycol), acrylic acid, acrylate polymer,

A further object of the invention is to disclose the mesh comprising at least one intermediate layer.

A further object of the invention is to disclose the mesh comprising a plurality of open pores; he open pores are of sized selected from the group consisting of 1-10 μm, 10-20 μm, 20-30 μm, 30-40 μm, 40-50 μm, 50-60 μm, 60-70 μm, 70-80 μm, 80-90 μm, 90-100 μm, and any combination thereof,

A further object of the invention is to disclose the method of repairing a tissue aperture within a wall of a mammal internal cavity. The aforethe method comprises the steps of
(a) providing an implantable mesh of a laminar extra-cellular-matrix-like structure comprising a first layer characterized by a predetermined porosity and a substantially non-porous second layer; the first layer is adapted to surgically adhere to a cavity wall in need of repair such that wall tissues infiltrate thereinto while the second layer characterized by non-adhesion and adapted for non-traumatic contact to mammal viscera;
(b) inserting the mesh into a mammal cavity; and
(c) tightly attaching the mesh to a mammal cavity wall;
(d) infiltrating the wall tissues into the first layer; and
(e) non-traumatically contacting the mammalian viscera by means of the second layer;

The aforesaid method further comprises the steps of biodegrading said first layer and permanently residing said second layer on said wall with tissue integration therebetween.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a microphotograph of the artificial nano-fiber mesh;

FIG. 2 is a photograph of the microsection of the two-layer mesh;

FIG. 3 is a microphotograph of a Novamesh hernia mesh at two weeks post implantation;

FIGS. 4a and 4b are scanning electron microscope views of the pristine non-adhesive NovaMesh layer before and after implantation, and

FIGS. 5a and 5b are environmental scanning electron microscope views of the native porcine peritoneal tissue and porcine peritoneal tissue at 1 month after implantation;

### DETAILED DESCRIPTION OF THE INVENTION

The following description is provided, alongside all chapters of the present invention, so as to enable any person skilled in the art to make use of said invention, and sets forth the best modes contemplated by the inventor of carrying out this invention. Various modifications, however, are adapted to remain apparent to those skilled in the art, since the generic principles of the present invention have been defined specifically to provide a fibrous mesh surgically implantable into mammal internal cavity and a method of repairing a tissue aperture.

The term ‘hernia’ hereinafter refers to a protrusion of a tissue, structure, or part of an organ through the muscular tissue or the membrane by which it is normally contained. The hernia has three parts: the orifice through which the aforesaid hernia herniates, the hernial sac, and contents of the aforesaid sac.

The term ‘extra-cellular matrix (ECM)’ hereinafter refers to an extracellular part of animal tissue that usually provides structural support to the cells in addition to performing various other important functions. The extracellular matrix is the defining feature of connective tissue in animals.

The term ‘viscus’ (plural: viscera) hereinafter refers to an internal organ of an animal (including humans), in particular an internal organ of the thorax or abdomen.

The term ‘porosity of a porous medium’ hereinafter refers to a fraction of void space in the material, where the void may contain, for example, air or water. The porosity φ is defined by the ratio:

$$φ = \frac{{V}_{V}}{{V}_{T}}$$

where V_{V} is the volume of void-space (such as fluids) and V_{T} is the total or bulk volume of material, including the solid and void components. Porosity is a fraction between 0 and 1, typically ranging from less than 0.01 for solid granite to more than 0.5 for peat and clay.

The term ‘tissue integration’ hereinafter refers to a tissue-mesh interface characterized by long-term biological stability and mechanical solidity.

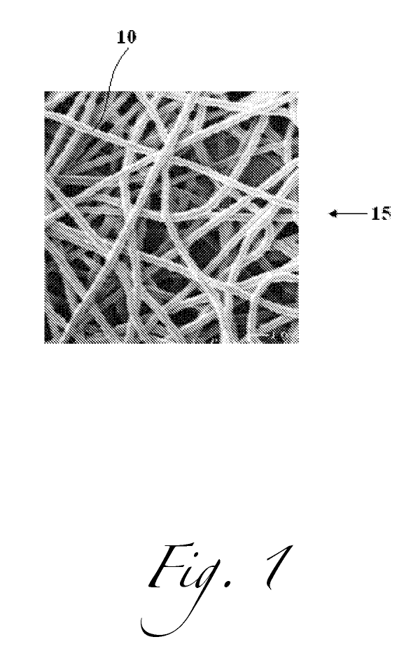

Reference is now made to FIG. 1, presenting an artificial nano-fiber mesh 15 produced by means of electrospinning. The polymer nano-fibers 10 form ECM-like structure. The aforesaid artificial mesh when surgically attached to herniated wall of a mammal wall, e.g. a herniated human abdominal wall, enables wall tissues to infiltrate into the mesh. It should be emphasized that EMC-like structures provide open pores (gaps between nano-fibers 10) with no real pore walls as for the pores formed in other known implantable materials. Thus, the artificial meshes of similar structure are applicable for hernia repair more effectively.

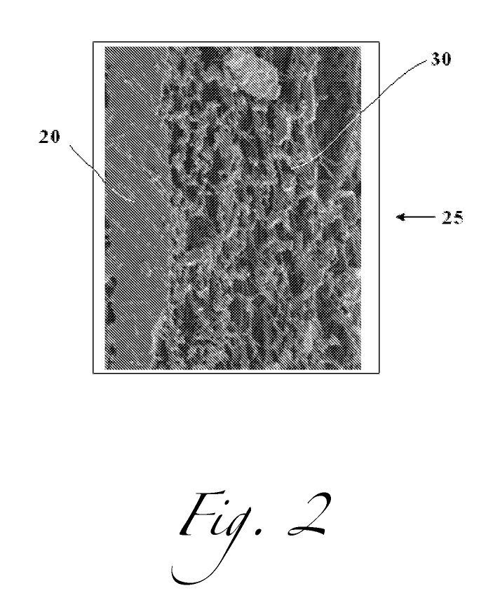

Reference is now made to FIG. 2, showing a microsection of a two-layer mesh 25 usable for repairing a tissue aperture, e.g. for repairing a hernia, specifically, an inguinal hernia, a femoral hernia, an umbilical hernia, a diaphragmatic hernia or an incisional hernia. The aforesaid mesh comprises two layers 20 and 30. As seen in FIG. 2, the layer 20 is characterized by a high value of porosity while the layer 30 is non-porous and has a smooth outer surface. In accordance with the preferable embodiment of the current invention, the layer 20 is provided with the porosity ranged between 72 and 80%, and the pore sizes of 10-100 μm, as measured using a capillary flow porometer. The mesh comprises a plurality of open pores. The meshes with the open pores of sizes selected from the group consisting of 10-20 μm, 20-30 μm, 30-40 μm, 40-50 μm, 50-60 μm, 60-70 μm, 70-80 μm, 80-90 μm, 90-100 μm, and any combination thereof are in the scope of the current invention,

The two-layer mesh 25 is surgically implanted into a mammal cavity to be attached to a herniated cavity wall, e.g. a human abdominal wall, so that the layer 20 adheres to wall tissues while the layer 30 is in contact to the viscera. The highly porous layer 20 enables the abdominal wall tissues to infiltrate thereinto and more reliably fixate the mesh 25 at the hernia. More extended infiltration of the wall tissue into the layer 20 reduces a risk of recrudescence.

As said above, the layer 30 has the smooth surface and provides non-traumatic contact to the viscera. The non-porous hydrophobic surface of the layer 30 provides inadhesion relative to the viscera that prevents trauma of internals. Tissues of the internals slide over the layer 30 and do not penetrate thereinto. An additional anti-traumatic effect is achieved by high elastic property of the electrospinningly made at least two-layer mesh. The electrospinning technology provides implantable materials characterized by the elasticity reaching a value of 500%. Thus, the implanted mesh 25 becomes an integral part of the abdominal wall and is deformed therewith.

The proposed mesh 25 is applicable by means of minimally invasive methods. The aforesaid mesh can be inserted into the human abdominal cavity through a lumen of an endo-/laparoscope in a folded form. The mesh 25 unbends in the abdominal cavity due to an inherent property of shape memory.

In accordance with the current invention, the layer 20 is made of a material providing cellular adhesion such as hydrophilic materials, e.g. materials from the PUR family, biological materials e.g. natural ECM components e.g. collagen, fibrin, fibronectin, vitronectin and laminin and their composites and all material/protein bearing cellular adhesion peptides, natural or synthetic, such as RGD (arginine-glycine-aspartic acid), RGDS (arf-gly-asp-ser), YIGSR (Tyr-Ile-Gly-Ser-Arg) and/or CDPGYIGSR (Cys-Asp-Pro-Gly-Tyr-Ile-Gly-Ser-Arg). Also cell adherence may be induced or enhanced by addition of materials which promote cellular electrostatic attraction such as poly-lysine. Also tissue ingrowth can be promoted and/or enhanced by addition and/or linking biochemicals known to promote/induce cell proliferation e.g. growth factors. Also viability of the infiltrated tissues can be enhanced by addition and/or linking biochemicals known to promote and/or enhance angiogenesis or neo-vascularization. As the pore size is thought to be important for cell migration and tissue infiltration, it may be controlled using degradable and/or bio absorbable and/or soluble materials combined with the main structural material, e.g. PLA, PGA, and PEC.

The layer 30 is made of material known for their anti-adhesion properties, such as PTFE, PVDF and all fluor based polymer, and/or hydrophobic materials, PE, PP, Delrin, silicone rubber, and hydrophilic materials such as poly(ethylene glycol), acrylic acid used alone or a composite of various materials and/or interpenetrating polymer networks and/or copolymers. Also biological materials known to “repel” cells and to avoid their attachment, and their derivatives, such as albumin or heparin may be used for this purpose. The structure of the material may be a film layer or an electro-spun nano-fiber structure with very low porosity and/or nanometric pore size, or a gel containing the raw material and water prepared during the device production or at the theater of surgery or in situ.

In accordance with the current invention, the fibrous mesh surgically is implanted into human internal cavity, e.g the abdominal cavity. The aforesaid mesh has a laminar extra-cellular-matrix-like structure and comprises the layer 20 characterized by a porosity effective for human tissue infiltration thereinto and the substantially non-porous layer 30.

The layer 20 is adapted to be surgically adhered to the abdominal wall such that wall tissues infiltrate into the layer 20 while the layer 30 characterised by non-adhesion and adapted for non-traumatic contact to mammal viscera and omentum.

The method of repairing a tissue aperture is in the scope of the current invention; The repairing method comprises the steps of (a) providing an implantable mesh of a laminar extra-cellular-matrix-like structure comprising the layer 20 characterized by a predetermined porosity and the substantially non-porous layer 30; (b) inserting the mesh into a human cavity; and (c) tightly attaching the mesh to a mammal cavity wall.

The step of attaching the mesh further comprises a step of attaching the layer 20 to a human cavity wall such that wall tissues are able to infiltrate thereinto and the layer 30 is in non-traumatic contact to mammal viscera and omentum.

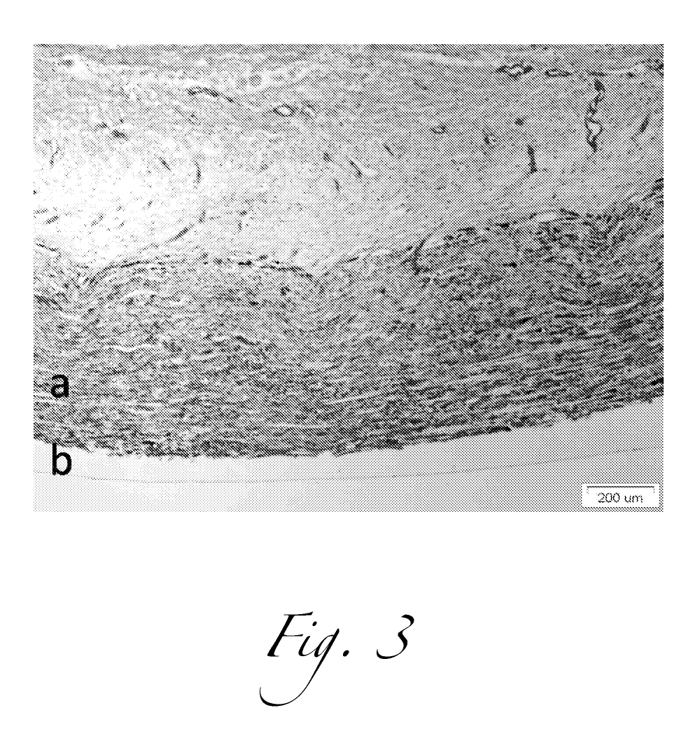

Reference is now made to FIG. 3, presenting a microphotograph of a Novamesh hernia mesh at two weeks post implantation. Specifically, (a) refers to a highly porous layer infiltrated by abdominal tissue. The cells gradually biodegrade the polycarbonate urethane nanofibers. (b) is a highly stable non-biodegraded filmy polycarbonate urethane layer (H&E staining).

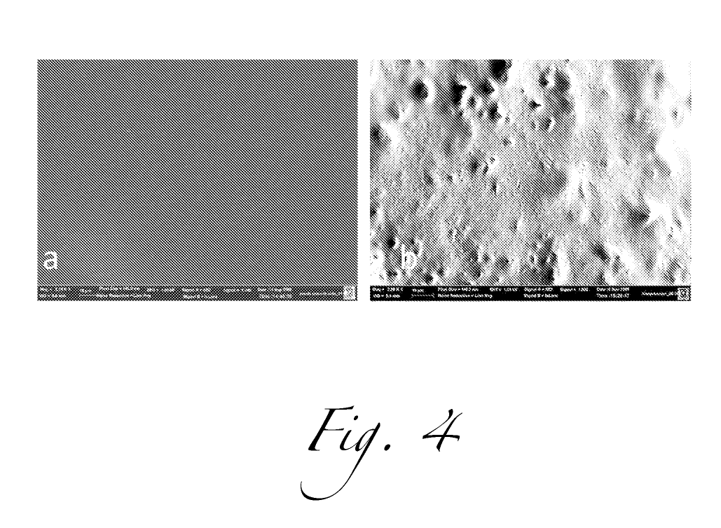

Reference is now made to FIG. 4, presenting a scanning electron microscope view of the pristine non-adhesive NovaMesh layer (a) and the porcine neo-peritoneal tissue covering the NovaMesh non-adhesive layer at 1 month after implantation

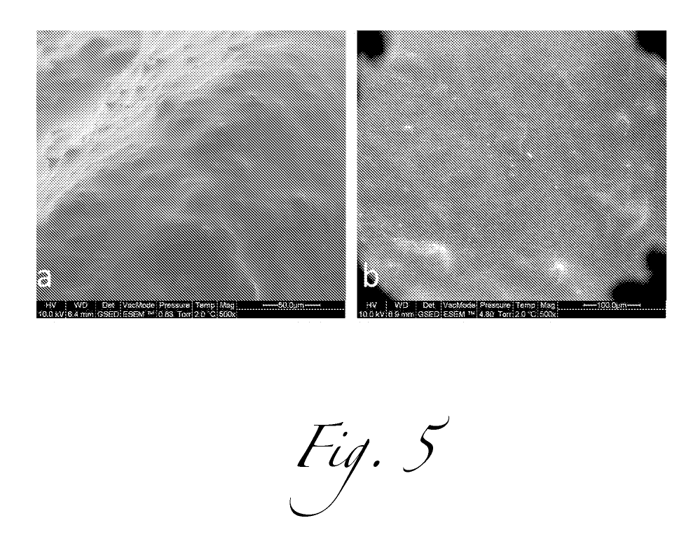

Reference is now made to FIG. 5, showing an environmental scanning electron microscope view the native porcine peritoneal tissue (a) and the porcine peritoneal tissue at 1 month after implantation. It should be emphasized that both tissues are structurally similar.

1. A fibrous mesh surgically implantable into a mammal internal cavity; said mesh has a laminar extra-cellular-matrix-like structure; said mesh comprises a first layer characterized by a porosity effective for mammal tissue infiltration into said first layer and a substantially non-porous bio-stable second layer; said first layer is adapted to surgically adhere to a cavity wall in need of repair such that wall tissues infiltrate thereinto while said second layer is characterized by non-adhesion and adapted for non-traumatic contact to mammal viscera and omentum; wherein said first layer is biodegradable and said second layer is biostable and tissue-supporting with said cavity wall.

2. The mesh according to claim 1, wherein said mesh is effectively elastic for non-interfering with a repaired mammal cavity wall.

3. The mesh according to claim 1, wherein said mammal is a human.

4. The mesh according to claim 1, wherein said mesh comprises electrospun fibres.

5. The mesh according to claim 1, wherein said electrospun fibers are of nanometric size.

6. The mesh according to claim 1, wherein said first layer is made of a material selected from the group consisting of polyurethane, collagen, fibrin, fibronectin, vitronectin, laminin, protein further comprising cellular adhesion peptides, protein comprising CDPGYIGSR (Cys-Asp-Pro-Gly-Tyr-Ile-Gly-Ser-Arg) peptide linked polymer, arginine-glycine-aspartic acid peptide linked polymer, RGDS (arf-gly-asp-ser) peptide linked polymer, YIGSR (Tyr-Ile-Gly-Ser-Arg) peptide linked polymer, and any combination thereof.

7. The mesh according to claim 1, wherein said protein comprises at least one of component selected from the group consisting of arginine-glycine-aspartic acid-rich sequences, RGDS (arf-gly-asp-ser)-rich sequences, YIGSR (Tyr-Ile-Gly-Ser-Arg)-rich sequences, CDPGYIGSR (Cys-Asp-Pro-Gly-Tyr-Ile-Gly-Ser-Arg)-rich sequences and any combination thereof.

8. The mesh according to claim 1, wherein said second layer is made of a material selected from the group consisting of polytetrafluorethylene, fluor based polymer, polyvinylidene fluoride, a hydrophobic material, polyester, polypropylene, polyformaldehyde, silicone rubber, poly(ethylene glycol), acrylic acid, acrylate polymer,

9. The mesh according to claim 1, wherein said mesh comprises at least one intermediate layer.

10. The mesh according to claim 1, wherein said mesh comprises a plurality of open pores; said open pores are of sized selected from the group consisting of 1-10 μm, 10-20 μm, 20-30 μm, 30-40 μm, 40-50 μm, 50-60 μm, 60-70 μm, 70-80 μm, 80-90 μm, 90-100 μm, and any combination thereof,

11. A method of repairing a tissue aperture within a wall of a mammal internal cavity; said method comprises the steps of
(a) providing an implantable mesh of a laminar extra-cellular-matrix-like structure comprising a first layer characterized by a predetermined porosity and a substantially non-porous second layer; said first layer is adapted to surgically adhere to a cavity wall in need of repair such that wall tissues infiltrate thereinto while said second layer characterized by non-adhesion and adapted for non-traumatic contact to mammal viscera and omentum;
(b) inserting said mesh into a mammal cavity; and
(c) tightly attaching said mesh to a mammal cavity wall;
(d) infiltrating said wall tissues into said first layer; and
(e) non-traumatically contacting said mammalian viscera by means of said second layer;
wherein said method further comprises the steps of biodegrading said first layer and permanently residing said second layer on said wall with tissue support therebetween.

12. The method according to claim 11 wherein said mesh is effectively elastic for non-interfering with to a repaired mammal cavity wall.

13. The method according to claim 11, wherein said aperture is a hernia.

14. The method according to claim 11, wherein said hernia is selected from the group consisting of an inguinal hernia, a femoral hernia, an umbilical hernia, a diaphragmatic hernia and an incisional hernia.

15. The method according to claim 11, wherein said mammal is a human.

16. The method according to claim 11, wherein said mesh comprises electrospun fibres.

17. The method according to claim 11, wherein said electrospun fibers are of nanometric size.

18. The method according to claim 11, wherein said first layer is made of a material selected from the group consisting of polyurethane, collagen, fibrin, fibronectin, vitronectin, laminin, protein further comprising cellular adhesion peptides, protein comprising CDPGYIGSR (Cys-Asp-Pro-Gly-Tyr-Ile-Gly-Ser-Arg) peptide linked polymer, arginine-glycine-aspartic acid peptide linked polymer, RGDS (arf-gly-asp-ser) peptide linked polymer, YIGSR (Tyr-Ile-Gly-Ser-Arg) peptide linked polymer, and any combination thereof.

19. The method according to claim 11,wherein said protein comprises at least one of component selected from the group consisting of arginine-glycine-aspartic acid-rich sequences, RGDS (arf-gly-asp-ser)-rich sequences, YIGSR (Tyr-Ile-Gly-Ser-Arg)-rich sequences, CDPGYIGSR (Cys-Asp-Pro-Gly-Tyr-Ile-Gly-Ser-Arg)-rich sequences and any combination thereof.

20. The method according to claim 11, wherein said second layer is made of a material selected from the group consisting of polytetrafluorethylene, fluor based polymer, polyvinylidene fluoride, a hydrophobic material, polyester, polypropylene, polyformaldehyde, silicone rubber, poly(ethylene glycol), acrylic acid, acrylate polymer,
